(19) United States
(12) Patent Application Publication
(10) Pub. No.: US 2004/0192928 A1
(43) Pub. Date: Sep. 30, 2004

(54) Novel compounds of the family of 3-alkyl-(4,5-diphenyl-imidazol-1-yl) and their use as soothing agents

(76) Inventors: Jean-Baptiste Galey, Aulnay-sous-Bois (FR); Maria Dalko, Yvette (FR); Yann Mahe, Morsang-sur-Orge (FR); Jacqueline Dumats, Villepinte (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/475,652

(22) PCT Filed: Apr. 24, 2002
(86) PCT No.: PCT/FR02/01411

(30) Foreign Application Priority Data
Apr. 24, 2001 (FR) 01/05497

(51) Int. Cl. C07D 233/61; C07D 233/54

(52) U.S. Cl. 548/311.1; 548/335.5; 548/341.1

(57) ABSTRACT

The invention concerns novel compounds of the family of 3-Alkyl-(4,5 diphenyl-imidazol-1-yl), their synthesis method and cosmetic, hygienic or pharmaceutical compositions containing them. The invention also concerns the use, in a physiologically acceptable medium, in or for preparing a soothing composition, of at least a compound of the family of 3-Alkyl-(4,5 diphenyl-imidazol-1-yl). The invention further concerns the use, in a physiologically acceptable medium, in or for producing a composition, of at least a compound of the family of 3-Alkyl-(4,5 diphenyl-imidazol-1-yl), the compound or the composition being designed to soothe skin troubles such as sensitive skins, discomfort, gnawing, itching, irritations, red spots, heat and/or flush sensations, advantageously sensitive and/or irritable and/or reactive and/or allergic symptoms of the skin and/or the scalp and/or mucosa. Finally, the invention concerns a soothing cosmetic method using such

The present invention relates to novel compounds of the 3-alkyl(4,5-diphenylimidazol-1-yl) family, to a process for synthesizing them and to cosmetic, hygiene or pharmaceutical compositions containing them.

The invention also relates to the use of at least one compound of the 3-alkyl(4,5-diphenylimidazol-1-yl) family, in a physiologically acceptable medium, in or for the preparation of a calmative composition.

The invention also relates to the use of at least one compound of the 3-alkyl(4,5-diphenylimidazol-1-yl) family, in a physiologically acceptable medium, in or for the preparation of a composition, the compound or the composition being intended to soothe skin disturbances such as sensitive skin, discomfort, tautness, itching, irritation, redness, hot sensations and/or sensations of inflammation, advantageously the symptoms of sensitive and/or irritable and/or reactive and/or intolerant skin and/or scalp and/or mucous membranes.

The invention also relates to a calmative cosmetic process using such a composition.

It is known that human beings are subject to phenomena of an attacking nature characterized, for example, by a sensation of skin discomfort, tautness, itching, irritation, sensations of hot skin or redness.

Novel compounds for soothing these types of attack have been sought for many years in the cosmetics industry.

Even though solutions have already been proposed, it is always advantageous to have available novel products with calmative activity, especially for minor skin disturbances, for instance sensations of inflammation, discomfort, tautness, itching, redness, a hot sensation, the symptoms of sensitive and/or irritable and/or reactive and/or intolerant skin and/or scalp and/or mucous membranes, and dry patches.

For the purposes of the present invention, the expression “skin disturbances”, means sensations of inflammation, discomfort, tautness, itching, irritation, redness, a hot sensation, the symptoms of sensitive and/or irritable and/or reactive and/or intolerant skin and/or scalp and/or mucous membranes, and dry patches.

Sensitive and/or irritable and/or reactive and/or intolerant skin and/or scalp and/or mucous membranes have been defined and characterized by the Applicant in patent EP 0 680 749. They are characterized by symptoms such as, for example, subjective signs that are essentially dysaesthetic sensations, i.e. sensations experienced in an area of skin, for instance stinging, tingling, itching or pruritus, burning, inflammation, discomfort, tautness, etc.

In addition, sensitive skin is not allergic skin and the symptoms of sensitive skin are distinguished from inflammation by the absence of oedema.

The aim of the present invention is thus to provide novel products, which are readily synthetically available, which have calmative activity while at the same time not having any appreciable side effects.

It is known in the literature, especially from patent applications WO 95/03297 and WO 95/02591, that derivatives of the 5-arylimidazole type are capable of blocking the release of certain inflammatory cytokines. Similarly, 1-[3-(4-morpholinyl)propyl]-4-(4-fluorophenyl)-5-(4-pyridyl)imidazole has already been described for the same activity in the reference J. Med. Chem. 1996, 39, 3929-3937.

All these derivatives of 5-arylimidazole type have very powerful pharmacological activities that position them as medicinal products.

In addition, certain compounds of the 3-alkyl(4,5-diphenylimidazol-1-yl) family are described in U.S. Pat. No. 3,759,946.

The Applicant has now discovered novel compounds of the 3-alkyl(4,5-diphenylimidazol-1-yl) family corresponding to formula (I), which are synthetically readily available.

A first subject of the invention relates to novel compounds of the 3-alkyl(4,5-diphenylimidazol-1-yl) family corresponding to formula (I) below:

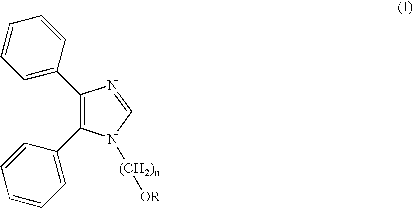

in which:

n is equal to 3, 4 or 5,

R represents, with the exception of an unsubstituted linear C₁-C₄ alkyl radical and/or an unsubstituted aryl radical:

a linear or branched C₁-C₈ alkyl radical, optionally substituted with at least one aryl and/or aralkyl radical and/or a hydroxyl radical (—OH) and/or a radical —OR₁ and/or a radical —SR₁ and/or a radical —NR₁R₂ and/or a heterocycle, for which R₁ and R₂ represent a linear or branched C₁-C₄ alkyl radical,

an aryl or alkylaryl or arylalkyl radical or a heterocycle, optionally substituted with at least one alkyl radical and/or a hydroxyl radical (—OH) and/or a radical —OR₁, and/or a radical —SR₁ and/or a radical —NR₁R₂, for which R₁ and R₂ represent a linear or branched C₁-C₄ alkyl radical, a phenyl radical or a benzyl radical.

The invention also relates to the optical and/or geometrical isomers and also the physiologically acceptable salts of the compounds corresponding to formula (I), alone or as a mixture in all proportions.

According to the invention, the expression “linear or branched C₁-C₄ and C₁-C₈ alkyl radical” means acyclic radicals derived from the removal of a hydrogen atom in the molecule of a linear or branched hydrocarbon containing from 1 to 4, or from 1 to 8, carbon atoms, and in particular methyl, ethyl, propyl, isopropyl, butyl, isobutyl, tert-butyl, pentyl, hexyl and heptyl radicals, and also the corresponding positional isomers thereof.

According to the invention, the term “heterocycle” means a sequence of atoms closed on itself and comprising at least one ring member (hetero atom) that is different from the others. According to the invention, the heterocycle may or may not be aromatic.

Among the compounds corresponding to formula (I) that are most particularly preferred are:

3-benzyloxypropyl(4,5-diphenylimidazol)-1-yl;

4-benzyloxybutyl(4,5-diphenylimidazol)-1-yl;

5-benzyloxypentyl(4,5-diphenylimidazol)-1-yl;

3-(2-tetrahydro-2H-pyranoxy)propyl(4,5-diphenyl-imidazol)-1-yl;

4-(2-tetrahydro-2H-pyranoxy)butyl(4,5-diphenyl-imidazol)-1-yl;

5-(2-tetrahydro-2H-pyranoxy)pentyl(4,5-diphenyl-imidazol)-1-yl;

3-(2-methoxyethoxy)propyl(4,5-diphenylimidazol)-1-yl;

4-(2-methoxyethoky)butyl(4,5-diphenylimidazol)-1-yl;

5-(2-methoxyethoxy)pentyl(4,5-diphenylimidazol)-1-yl;

3-(2-ethoxyethoxy)propyl(4,5-diphenylimidazol)-1-yl;

4-(2-ethoxyethoxy)butyl(4,5-diphenylimidazol)-1-yl;

5-(2-ethoxyethoxy)pentyl(4,5-diphenylimidazol)-1-yl;

3-(2-propyloxyethoxy)propyl(4,5-diphenylimidazol)-1-yl;

4-(2-propyloxyethoxy)butyl(4,5-diphenylimidazol)-1-yl ;

5-(2-propyloxyethoxy)pentyl(4,5-diphenylimidazol)-1-yl;

3-(methoxymethoxy)propyl(4,5-diphenylimidazol)-1-yl;

4-(methoxymethoxy)butyl(4,5-diphenylimidazol)-1-yl;

5-(methoxymethoxy)pentyl(4,5-diphenylimidazol)-1-yl;

3-(ethoxymethoxy)propyl(4,5-diphenylimidazol)-1-yl;

4-(ethoxymethoxy)butyl(4,5-diphenylimidazol)-1-yl;

5-(ethoxymethoxy)pentyl(4,5-diphenylimidazol)-1-yl;

3-(propyloxymethoxy)propyl(4,5-diphenylimidazol)-1-yl;

4-(propyloxymethoxy)butyl(4,5-diphenylimidazol)-1-yl;

5-(propyloxymethoxy)pentyl(4,5-diphenylimidazol)-1-yl;

3-(3-methoxypropyloxy)propyl(4,5-diphenylimidazol)-1-yl;

4-(3-methoxypropyloxy)butyl(4,5-diphenylimidazol)-1-yl;

5-(3-methoxypropyloxy)pentyl(4,5-diphenylimidazol)-1-yl;

3-(3-ethoxypropyloxy)propyl(4,5-diphenylimidazol)-1-yl;

4-(3-ethoxypropyloxy)butyl(4,5-diphenylimidazol)-1-yl;

5-(3-ethoxypropyloxy)pentyl(4,5-diphenylimidazol)-1-yl;

3-(3-propyloxypropyloxy)propyl(4,5-diphenylimidazol)-1-yl;

4-(3-propyloxypropyloxy)butyl(4,5-diphenylimidazol)-1-yl;

5-(3-propyloxypropyloxy)pentyl(4,5-diphenylimidazol)-1-yl;

3-(2,3,4-trimethoxybenzyloxy)propyl(4,5-diphenylimidazol)-1-yl;

4-(2,3,4-trimethoxybenzyloxy)butyl(4,5-diphenylimidazol)-1-yl;

5-(2,3,4-trimethoxybenzyloxy)pentyl(4,5-diphenylimidazol)-1-yl;

3-(3,4-dimethoxybenzyloxy)propyl(4,5-diphenylimidazol)-1-yl;

4-(3,4-dimethoxybenzyloxy)butyl(4,5-diphenylimidazol)-1-yl;

5-(3,4-dimethoxybenzyloxy)pentyl(4,5-diphenylimidazol)-1-yl;

3-(3-methoxybenzyloxy)propyl(4,5-diphenylimidazol)-1-yl;

4-(3-methoxybenzyloxy)butyl(4,5-diphenylimidazol)-1-yl;

5-(3-methoxybenzyloxy)pentyl(4,5-diphenylimidazol)-1-yl;

3-(4-methoxybenzyloxy)propyl(4,5-diphenylimidazol)-1-yl;

4-(4-methoxybenzyloxy)butyl(4,5-diphenylimidazol)-1-yl;

5-(4-methoxybenzyloxy)pentyl(4,5-diphenylimidazol)-1-yl;

3-(3-pyridylmethoxy)propyl(4,5-diphenylimidazol)-1-yl;

4-(3-pyridylmethoxy)butyl(4,5-diphenylimidazol)-1-yl;

5-(3-pyridylmethoxy)pentyl(4,5-diphenylimidazol)-1-yl;

3-(4-benzyloxy-3-methoxybenzyloxy)propyl(4,5-diphenylimidazol)-1-yl;

4-(4-benzyloxy-3-methoxybenzyloxy)butyl(4,5-diphenylimidazol)-1-yl;

5-(4-benzyloxy-3-methoxybenzyloxy)pentyl(4,5-diphenylimidazol)-1-yl;

3-(1-naphthalenemethoxy)propyl(4,5-diphenylimidazol)-1-yl;

4-(1-naphthalenemethoxy)butyl(4,5-diphenylimidazol)-1-yl;

5-(1-naphthalenemethoxy)pentyl(4,5-diphenylimidazol)-1-yl;

The compounds of formula (I) that are preferred are those for which:

n is equal to 3 or 4;

R denotes a benzyl radical.

Advantageously, the compounds corresponding to formula (I) are chosen from the following compounds:

3-benzyloxypropyl(4,5-diphenylimidazol)-1-yl;

4-benzyloxybutyl(4,5-diphenylimidazol)-1-yl;

The compounds corresponding to formula (I) are obtained in a single step from 4,5-diphenylimidazole, which is a commercial product, and from a halo derivative.

Thus, the process for synthesizing the compounds of the 3-alkyl(4,5-diphenylimidazol-1-yl) family according to the invention has the advantage of allowing quick and easy access to the said compounds compared with the existing synthetic processes described elsewhere (WO 95/03297, WO 95/02591, U.S. Pat. No. 3,759,946 and J. Med. Chem. 1996, 39, 3929), due to the fact that they are obtained in a single synthetic step.

According to the process of the invention, the formation of the compounds of the 3-alkyl(4,5-diphenylimidazol-1-yl) family of formula (I) results from the nucleophilic substitution of a halogen, derived from an alkyl halide, with the 4,5-diphenylimidazole anion.

Thus, a second subject of the invention relates to a process for preparing the compounds of formula (I) as defined above, from 4,5-diphenylimidazole.

According to a first embodiment, the process for preparing the compounds of formula (I) is characterized in that it consists essentially of the following steps:

a) dissolving 4,5-diphenylimidazole in an aprotic organic solvent preferably chosen from acetonitrile, acetone, tetrahydrofuran (THF) and dimethylformamide (DMF), advantageously acetonitrile;

b) adding an organic or mineral base, preferably a mineral base, advantageously chosen from potassium carbonate (K₂CO₃), sodium carbonate (Na₂CO₃) and calcium carbonate (Ca₂CO₃), most preferably K₂CO₃, in an amount of between 1 and 10 equivalents, preferably between 1 and 3 equivalents and advantageously 2 equivalents of base;

c) adding an alkyl halide in an amount of between 1 and 5 equivalents, advantageously 1.5 equivalents;

d) heating to a temperature of between 50° C. and 85° C., preferably between 70° C. and 85° C., for a period of between 6 hours and 48 hours, advantageously between 12 hours and 30 hours, preferably 24 hours;

e) evaporating the reaction medium to dryness under vacuum after cooling to room temperature;

f) dissolving the residue in water;

g) extracting the aqueous solution with an organic solvent chosen from ethyl acetate, dichloromethane and diethyl ether, preferably ethyl acetate;

h) drying and evaporating the organic phase to dryness;

i) optionally purifying the residue.

According to a second embodiment, the process for preparing the compounds of formula (I) is characterized in that it consists essentially of the following steps:

a) dissolving 4,5-diphenylimidazole in a dipolar aprotic solvent preferably chosen from dimethylformamide (DMF), dimethyl sulphoxide (DMSO) and dimethylacetamide (DMAC), advantageously DMF;

b) adding under an inert atmosphere an organic or mineral base, preferably a mineral base, advantageously chosen from sodium hydride (NaH) and butyllithium (BuLi), most preferably NaH, in an amount of between 0.9 and 1.5 equivalents, preferably between 1.0 and 1.1 equivalents, advantageously 1.0 equivalent;

c) stirring the reaction medium at a temperature of between 25° C. and 100° C., preferably between 40° C. and 60° C., advantageously at 50° C., for a period of between 30 minutes and 10 hours, advantageously between 1 hour and 2 hours, preferably for 1 hour;

d) adding an alkyl halide in an amount of between 1 and 3 equivalents, preferably between 1 and 2 equivalents, advantageously 1.2 equivalents;

e) adding potassium iodide or sodium iodide in an amount of between 1 and 3 equivalents, preferably between 1 and 2 equivalents, advantageously 1.2 equivalents;

f) heating at a temperature of between 25° C. and 100° C., preferably at 50° C., for a period of between 5 hours. and 24 hours, advantageously between 10 hours and 20 hours, preferably for 15 hours;

g) taking up the reaction medium in an organic solvent chosen from dichloromethane and chloroform, preferably dichloromethane;

h) washing the organic phase with water and bicarbonate, drying it and then evaporating it to dryness;

j) optionally purifying the residue.

The purification methods that may optionally be carried out at the end of the processes according to the invention are performed according to standard methods used in organic synthesis.

According to the invention, the term “inert atmosphere” means argon or nitrogen, and the term “room temperature” means a temperature of between 15° C. and 25° C.

Detailed examples of the preparation of the compounds according to the invention are given later in the examples.

A third subject of the invention relates to a composition that comprises at least one of the compounds corresponding to formula (I) defined above.

Needless to say, the composition according to the invention may comprise the compounds of formula (I) alone or as mixtures in all proportions.

The amount of compounds of formula (I) contained in the composition according to the invention obviously depends on the desired effect and may thus vary within a wide range.

To give an order of magnitude, the composition of the invention may contain at least one compound of formula (I) in an amount representing from 0.001% to 20% of the total weight of the composition and preferably in an amount representing from 0.01% to 5% of the total weight of the composition.

The composition according to the invention may be intended for cosmetic or pharmaceutical and particularly dermatological application.

Preferably, the composition according to the invention is intended for cosmetic application.

The composition may be ingested or applied to the skin (to any area of body skin), the hair, the nails or mucous membranes (buccal, jugal, gingival, genital or conjunctival membranes).

Depending on the mode of administration, the composition according to the invention may be in any presentation form normally used, particularly in cosmetology.

A preferred composition according to the invention is a cosmetic composition intended for topical application.

The composition according to the invention contains a physiologically acceptable medium, i.e. a medium that is compatible with the skin, the lips, the scalp, mucous membranes, the eyes and/or the hair.

According to a fourth subject of the invention, at least one compound of the 3-alkyl(4,5-diphenylimidazol-1-yl) family corresponding to formula (I), as defined in the text hereinabove, may be combined with products with an irritant side effect commonly used in cosmetics or pharmaceutics, which products are occasionally cosmetic or pharmaceutical active agents. The presence of a compound of formula (I) in a cosmetic or pharmaceutical composition comprising a product with an irritant effect allows this irritant effect to be greatly attenuated, or even eliminated.

This also makes it possible to increase the amount of product with an irritant side effect compared with the amount of product normally used, for the purpose of improved efficacy.

Thus, the invention relates more particularly to a cosmetic composition, characterized in that it comprises, in a cosmetically or pharmaceutically acceptable medium, at least one product with an irritant side effect and at least one compound of the 3-alkyl(4,5-diphenylimidazol-1-yl) family corresponding to formula (I).

Examples of products with an irritant side effect that may be mentioned include surfactants (ionic or nonionic), preserving agents, organic solvents or active agents, for instance α-hydroxy acids (citric acid, malic acid, glycolic acid, tartaric acid, mandelic acid and lactic acid), β-hydroxy acids (salicylic acid and its derivatives), α-keto acids, β-keto acids, retinoids (retinol, retinal and retinoic acid), anthralins (dioxyanthranol), anthranoids, peroxides (especially benzoyl peroxide), lithium salts, antimetabolites, certain hair dyes or hair colorants (para-phenylenediamine and its derivatives, and aminophenols), fragrancing alcoholic solutions (fragrances, eaux de toilette, aftershave and deodorants), antiperspirants (certain aluminium salts), hair-removing or permanent-waving active agents (thiols) and depigmenting active agents (hydroquinone).

The Applicant has now discovered that the novel compounds of the 3-alkyl(4,5-diphenylimidazol-1-yl) family corresponding to formula (I) as defined above have calamative activity.

U.S. Pat. No. 3,759,946 describes the use of certain compounds of the 3-alkyl(4,5-diphenylimidazol-1-yl) family in the case of allergic diseases, urticaria and skin rashes.

Patent JP 44 029 199 reports antiirritant activity for certain compounds of the 3-alkyl(4,5-diphenylimidazol-1-yl) family.

The Applicant has been able, surprisingly and unexpectedly, to demonstrate superior antiirritant activities for the compounds of formula (I) according to the invention compared with those of the compounds described in U.S. Pat. No. 3,759,946.

Specifically, the comparative test presented in the examples of the present invention demonstrates that the measured percentage of inhibition (inhibition of the response of superficial epidermal cells to an irritant agent by assay of interleukin-8 secreted by NHEK cells after stimulation with PMA as irritant agent) is better for the compounds according to the invention compared with the reference compounds of the prior art.

On account of this antiirritant effect, the advantage of using the compounds according to the invention as calmatives may be readily appreciated.

Thus, the present invention also relates to the use in a physiologically acceptable medium, in or for the preparation of a calmative composition, of at least one compound of the 3-alkyl(4,5-diphenylimidazol-1-yl) family corresponding to formula (I) as defined above.

In addition, the compounds of the 3-alkyl(4,5-diphenylimidazol-1-yl) family corresponding to formula (I) also have the advantage of showing very little cytotoxicity (less than 20%). The evaluation of the cytotoxicity of the compounds was performed on normal human keratinocytes cultured in vitro by adding 3-(4,5-dimethylthiazol-2-yl)-2,5-diphenyl)tetrazolium bromide (MTT, 0.5 mg/ml) to the culture medium over the course of 2 hours, followed by spectrophotometric measurement of the formazan incorporated into the cells after 24 hours, according to the standard technique described by T. Mosmann (J. Immunological Methods; 65 (1983) 55-63).

Another advantage of the present invention is that of now having available compounds for gently soothing skin disturbances such as sensitive skin, discomfort, tautness, itching, irritation, redness, hot sensations and/or sensations of inflammation, which makes the use of these compounds compatible in cosmetic compositions, particularly topical compositions.

The expression “physiologically acceptable medium” means a medium that is compatible with the skin and/or mucous membranes and/or the nails and/or the hair.

Another subject of the invention relates to the use, in a physiologically acceptable medium, in or for the manufacture of a composition, of at least one compound of the 3-alkyl(4,5-diphenylimidazol-1-yl) family of formula (I) as defined above, the compound or the composition being intended to soothe skin disturbances chosen from sensitive skin, discomfort, tautness, itching, irritation, redness, hot sensations and/or sensations of inflammation, advantageously the symptoms of sensitive and/or irritable and/or reactive and/or intolerant skin and/or scalp and/or mucous membranes, particularly stinging, tingling, itching or pruritus, inflammation, discomfort and/or tautness.

Certain alopecias are associated with irritation of the scalp and/or symptoms such as discomfort, tautness, itching or pruritus, redness, hot sensations and/or sensations of inflammation. This is especially the case for androgenetic alopecia which results from a process that gives rise to irritation.

As a result, it may be appreciated that reducing the irritation of the scalp can represent a means for reducing and/or stabilizing natural hair loss in man.

Thus, another subject of the invention relates to the use, in a physiologically acceptable medium, in a cosmetic composition or -for the preparation of a pharmaceutical composition, of at least one compound of the 3-alkyl(4,5-diphenylimidazol-1-yl) family of formula (I) as defined above, the compound or the composition being intended to reduce and/or stabilize natural hair loss in man, advantageously androgenetic alopecia.

According to the invention, the compounds of the 3-alkyl(4,5-diphenylimidazol-1-yl) family corresponding to formula (I) that are preferably used according to the invention are those defined earlier in the text as being preferred.

Needless to say, according to the invention, the compounds of the 3-alkyl(4,5-diphenylimidazol-1-yl) family corresponding to formula (I) may be used alone or as a mixture in any proportion.

In the cosmetic treatment of the skin disturbances mentioned above, in particular the symptoms of sensitive and/or irritable and/or reactive and/or intolerant skin and/or scalp and/or mucous membranes, the cosmetic composition according to the invention is to be applied to the areas to be treated of an individual suffering from at least one of the said skin disturbances and/or symptoms and is optionally left in contact for from several minutes to several hours and is optionally rinsed off; this being for uses that may be repeated or renewed over treatment periods ranging from a few days to several months or even several years.

Thus, another subject of the present invention is a cosmetic process for treating the skin and/or the scalp and/or mucous membranes, which is intended to soothe at least one of the skin disturbances and/or one of the symptoms mentioned above, characterized in that it consists in applying to the skin and/or the scalp and/or mucous membranes a cosmetic composition comprising at least one compound corresponding to formula (I), in leaving the said composition in contact with the skin and/or mucous membranes and/or the scalp, and in optionally rinsing it off.

The Cosmetic treatment process of the invention advantageously applies to natural hair loss in man, and/or to sensitive and/or irritable and/or reactive and/or intolerant skin and/or scalp and/or mucous membranes.

The treatment process has the characteristics of a cosmetic process insofar as it can improve the aesthetics or comfort of the skin and/or mucous membranes and/or the scalp.

For topical application to the skin, the composition may especially be in the form of an aqueous or oily solution or a dispersion of lotion or serum type, emulsions of liquid or semi-liquid consistency of the milk type, obtained by dispersing a fatty phase in an aqueous phase (O/w) or conversely (W/O), or suspensions or emulsions of soft consistency of the aqueous or anhydrous cream or gel type, or alternatively microcapsules or microparticles or vesicular dispersions of ionic and/or nonionic type. These compositions are prepared according to the usual methods.

The composition according to the invention obviously comprises a cosmetically acceptable support and may be in any presentation form normally used for topical application, especially in the form of an aqueous, aqueous-alcoholic or oily solution, an oil-in-water or water-in-oil or multiple emulsion, an aqueous or oily gel, a liquid, pasty or solid anhydrous product, or a dispersion of oil in an aqueous phase using spherules, these spherules possibly being polymer nanoparticles such as nanospheres and nanocapsules or better still lipid vesicles of ionic and/or nonionic type.

This composition may be more or less fluid and may have the appearance of a white or coloured cream, a pomade, a milk, a lotion, a serum, a paste or a mousse. It may optionally be applied to the skin in the form of an aerosol. It may also be in solid form, for example in the form of a stick or a patch. It may be used as a care product, as a cleansing product, as a makeup product or as a simple deodorant product.

It may also be used for the hair in the form of aqueous, alcoholic or aqueous-alcoholic solutions, or in the form of creams, gels, emulsions or mousses, or alternatively in the form of aerosol compositions also comprising a pressurized propellant.

The composition according to the invention may also be a haircare composition, especially a shampoo, a hairsetting lotion, a medicated lotion, a styling cream or gel, a dye composition (especially for oxidation dyeing) optionally in the form of colouring shampoos, a restructuring lotion for the hair, a permanent-waving composition (especially a composition for the first stage of a permanent-waving operation), a lotion or gel for preventing hair loss, an antiparasitic shampoo, etc.

For the eyes, it may be in the form of drops, and for ingestion it may be in the form of capsules, granules, syrups or tablets.

The amounts of the various constituents of the compositions according to the invention are those conventionally used in the fields under consideration.

These compositions especially constitute cleansing creams, protective creams or care creams for the face, for the hands, for the feet, for the major anatomical folds or for the body (for example day creams, night creams, makeup-removing creams, foundation creams and antisun creams), fluid foundations, makeup-removing milks, protective or care body milks, aftersun milks, skincare lotions, gels or mousses, for instance cleansing lotions, antisun lotions, artificial tanning lotions, bath compositions, deodorizing compositions comprising a bactericidal agent, compositions for preventing hair loss, aftershave gels or lotions, and hair-removing creams.

The compositions according to the invention may also consist of solid preparations constituting cleansing soaps or bars.

The compositions may also be packaged in the form of an aerosol composition also comprising a pressurized propellant.

The composition may also be for buccodental use, for example a toothpaste. In this case, the composition may contain adjuvants and additives that are common for compositions for buccal use, and especially surfactants, thickeners, humectants, polishing agents such as silica, various active ingredients, for instance fluorides, in particular sodium fluoride, and optionally sweeteners, for instance sodium saccharinate.

When the composition is an emulsion, the proportion of the fatty phase may range from 5% to 80% by weight and preferably from 5% to 50% by weight relative to the total weight of the composition. The oils, waxes, emulsifiers and co-emulsifiers used in the composition in emulsion form are chosen from those conventionally used in the cosmetics field. The emulsifier and co-emulsifier are present in the composition in a proportion ranging from 0.3% to 30% by weight and preferably from 0.5% to 20% by weight relative to the total weight of the composition. The emulsion may also contain lipid vesicles.

When the composition is an oily solution or gel, the fatty phase may represent more than 90% of the total weight of the composition.

In a known manner, the cosmetic composition may also contain adjuvants that are common in cosmetics, such as hydrophilic or lipophilic gelling agents, hydrophilic or lipophilic additives, preserving agents, antioxidants, solvents, fragrances, fillers, screening agents, odour absorbers and dyestuffs. The amounts of these various adjuvants are those conventionally used in cosmetics, for example from 0.01% to 10% of the total weight of the composition. Depending on their nature, these adjuvants may be introduced into the fatty phase, into the aqueous phase and/or into the lipid spherules.

As oils or waxes that may be used in the invention, mention may be made of mineral oils (liquid petroleum jelly), plant oils (liquid fraction of karite butter, sunflower oil), animal oils (perhydrosqualene), synthetic oils (purcellin oil), silicone oils or waxes (cyclomethicone), fluoro oils (perfluoropolyethers), beeswax, carnauba wax or paraffin wax. Fatty alcohols and fatty acids (stearic acid) may be added to these oils.

As examples of emulsifiers that may be used in the invention, mention may be made of glyceryl stearate, polysorbate 60 and the mixture of PEG-6/PEG-32/glycol stearate sold under the name Tefose® 63 by the company Gattefosse.

As solvents that may be used in the invention, mention may be made of lower alcohols, especially ethanol and isopropanol, and propylene glycol.

As hydrophilic gelling agents that may be used in the invention, mention may be made of carboxyvinyl polymers (carbomer), acrylic copolymers such as acrylate/alkylacrylate copolymers, polyacrylamides, polysaccharides such as hydroxypropylcellulose, natural gums and clays, and, as lipophilic gelling agents, mention may be made of modified clays, for instance bentones, metal salts of fatty acids, for instance aluminium stearates, hydrophobic silica, ethylcellulose and polyethylene.

The composition may contain other hydrophilic active agents, for instance proteins or protein hydrolysates, amino acids, polyols, urea, allantoin, sugars and sugar derivatives, water-soluble vitamins, plant extracts and hydroxy acids.

Lipophilic active agents that may be used include retinol (vitamin A) and its derivatives, tocopherol (vitamin E) and its derivatives, essential fatty acids, ceramides, essential oils and salicylic acid and its derivatives.

According to the invention, the composition may combine at least one compound of formula (I) with other active agents. Among these active agents that may be mentioned, for example, are:

agents for improving the activity on regrowth of the hair and/or on stopping hair loss, and which have already been described for this activity, for instance nicotinic acid esters including, especially, tocopheryl nicotinate, benzyl nicotinate and C₁-C₆ alkyl nicotinates, for instance methyl or hexyl nicotinate, pyrimidine derivatives, for instance those described by the Applicant in patent EP 0 540 629, particularly 2,4-diaminopyrimidine 3-oxide or “Aminexil”, those described in U.S. Pat. Nos. 4,139,619 and 4,596,812, particularly 2,4-diamino-6-piperidinopyrimidine 3-oxide or “Minoxidil”, agents for promoting regrowth of the hair, for instance those described by the Applicant in patents EP-B-0 648 488 and EP-B-0 672 406, and 5-α-reductase inhibitors, for instance those described by the Applicant in patent application EP-A-0 964 852;

agents for modifying cutaneous differentiation and/or proliferation and/or pigmentation, such as retinoic acid and its isomers, retinol and its esters, vitamin D and its derivatives, oestrogens such as oestradiol, kojic acid or hydroquinone;

antibacterial agents such as clindamycin phosphate, erythromycin or antibiotics of the tetracycline class;

antiparasitic agents, in particular metronidazole, crotamiton or pyrethroids;

antifungal agents, in particular compounds belonging to the imidazole class, such as econazole, ketoconazole or miconazole or the. salts thereof, polyene compounds, such as amphotericin B, compounds of the allylamine family, such as terbinafine, or octopirox;

antiviral agents such as acyclovir;

antiinflammatory agents other than the compounds of formula (I) according to the invention, chosen, for trimeprazine or cyproheptadine;

keratolytic agents such as α- and β-hydroxycarboxylic acids or α- and β-keto carboxylic acids, and the salts, amides or esters thereof and more particularly hydroxy acids such as glycolic acid, lactic acid, salicylic acid, citric acid and fruit acids in general, and 5-n-octanoylsalicylic acid;

free-radical scavengers, such as α-tocopherol or its esters, superoxide dismutases, certain metal-chelating agents or ascorbic acid and its esters;

antiseborrhoeic agents such as progesterone;

antidandruff agents, for instance octopirox or zinc pyrithione;

antiacne agents, for instance retinoic acid or benzoyl peroxide;

extracts of plant and/or bacterial origin.

Other compounds may also be added to the above list, for instance Diazoxyde, Spiroxazone, phospholipids, for instance lecithin, linoleic acid, linolenic acid, salicylic acid, jasmonic acid and its derivatives described in French patent FR 2 581 542, for instance salicylic acid derivatives bearing an alkanoyl group containing from 2 to 12 carbon atoms in position 5 of the benzene ring, hydroxycarboxylic acids or keto carboxylic acids and the esters thereof, lactones and the corresponding salts thereof, anthralin, carotenoids, and eicosatetraenoic acid and eicosatrienoic acid or the esters and amides thereof.

According to one particular embodiment, the composition according to the invention also comprises at least one agent chosen from antibacterial agents, antiparasitic agents, antifungal agents, antiviral agents, antiinflammatory agents, antipruriginous agents, anaesthetics, keratolytic agents, free-radical scavengers, antiseborrhoeic agents, antidandruff agents, antiacne agents and/or agents for reducing cutaneous differentiation and/or proliferation and/or pigmentation, agents for improving the activity on regrowth of the hair. and/or on stopping hair loss, and extracts of plant and/or bacterial origin.

It may also be envisaged for the composition comprising at least one compound as defined above to be in liposomal form, as described especially in patent application WO 94/22468 filed on 13 Oct. 1994 by the company Anti Cancer Inc. Thus, the compound encapsulated in the liposomes may be selectively delivered to the hair follicle.

Examples, which should not be considered as limiting the scope of the invention in any way, will now be given by way of illustration.

### EXAMPLE 1

Parallel Synthetic Scheme:

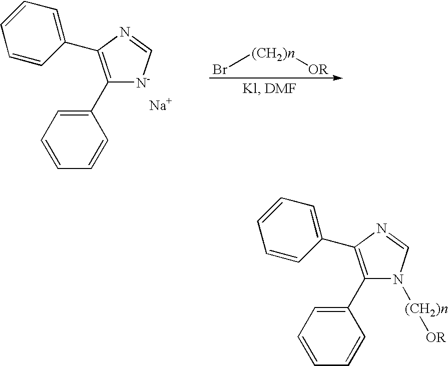

General Protocol:

A solution of the sodium salt of 4,5-diphenylimidazole is prepared by reacting 4,5-diphenylimidazole with one equivalent of sodium hydride in dimethylformamide (DMF) under argon at 50° C. for 1 hour. The solution is then divided among the tubes of a multi-well reaction block, at a rate of 3 mmol per tube. 1.2 molar equivalents of alkyl halide and of potassium iodide dissolved in DMF are then added to each tube and the mixture is heated at 50° C. for 15 hours.

Each reaction mixture is taken up in dichloromethane and washed twice with water and bicarbonate. The organic phases are dried over sodium sulphate and evaporated to dryness.

Each residue is then analysed by HPLC coupled to mass spectrometry (electron-spray ionization).

The Results are Collated in the Following Table:

| | | FINAL PRODUCT | |
|---|---|---|---|
| | | | Result- |
| | STARTING | | analysis |
| EXAMPLE | MATERIALS | Structure-name | HPLC ESI-MS |
| 1 | 4,5-diphenyl- imidazole + | 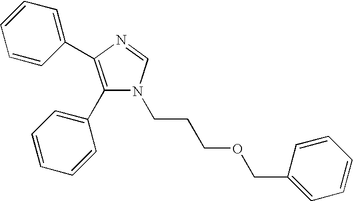 | [M + H]⁺ = 383 detected in the majority peak |
| | 3-benzyloxypropyl | 3-benzyloxypropyl- | |
| | bromide | (4,5-diphenylimidazol)-1-yl | |

### EXAMPLE 2

Inhibition of the response of superficial epidermal cells to an irritant agent by assay of the interleukin-8 secreted by normal human epidermal keratinocytes (NHEKs) after stimulation with phorbol 12-myristate 13-acetate (PMA) as irritant agent.

Principle and Aim of the Study:

This study uses the test developed by Wilmer [Wilmer, J. L. et al., J. invest. Dermatol., 102: 915-922 (1994)]. This test allows the evaluation of the antiirritant potential of various molecules, on a human keratinocyte (NHEK) cell line. In this test, an irritant situation is mimicked by exacerbating the production of IL-8 of the NHEKs by adding PMA to the culture medium, IL-8 being involved in initiating the keratinocyte irritation. The antiirritant effect of a molecule is then measured by means of its inhibitory action with respect to this exacerbated production.

Experimental Conditions:

Normal human epidermal keratinocytes (NHEKs) sold by the company Clonetics and distributed in France by the company TEBU are incubated in the presence of 160 nM of PMA for 24 hours at 37° C. In response to the PMA, the production of the chemotactic agent interleukin-8 (IL-8) is measured by enzymatic assay using an assay kit (Elisa D8050) sold by the company R&D. The absorbance values are measured using a microplate reader (MRX/Dynatech) according to the procedures supplied with. the assay kit. To evaluate the antiirritant protective effect of the various compounds, the production of the chemotactic marker IL-8 by the human keratinocytes is measured in the presence of various concentrations of the test compound.

The results are expressed as a percentage of the control values after subtracting the background noise and as a percentage of inhibition of these values obtained in the presence of the compounds.

Results

Comparative Test

| Test compounds | | % inhibition of the |
|---|---|---|
| (at 10 μM) | Structure | secretion of IL-8 |
| US 3 759 946 3-phenoxypropyl(4,5- diphenylimidazol)-1-yl | 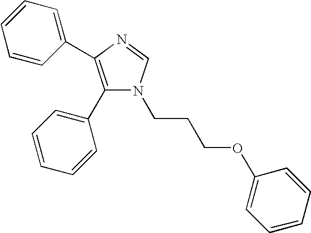 | 32 |
| Compound of Example 1 3-benzyloxypropyl (4,5- diphenylimidazol)-1-yl | 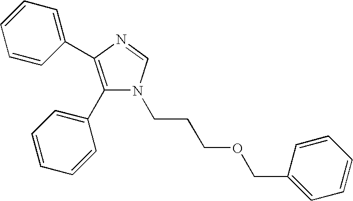 | 41 |

The results of the comparative test show that the measured percentage of inhibition (inhibition of the response of the superficial epidermal cells to an irritant agent by assay of the interleukin-8 secreted by NHEK cells after stimulation with PMA as irritant agent) is better for the compound according to the invention compared with the reference compounds of the prior art (U.S. Pat. No. 3,759,946).

These results thus demonstrate an anti-irritant effect of the compounds according to the invention that is better than that of the reference compounds of the prior art. As a result of this anti-irritant effect, the advantage of using the compounds according to the invention as calmatives may be readily appreciated.

### EXAMPLE 3

### Compositions

These compositions. were obtained by simple mixing of the various components.

Lotion:

| | |
|---|---|
| 3-Benzyloxypropyl(4,5-diphenylimidazol)-1-yl | 0.5% |
| Propylene glycol | 10.0% |
| Isopropyl alcohol qs | 100% |

1 ml of this lotion is applied to the scalp, at a frequency of once or twice a day.

Gel:

| | |
|---|---|
| Chimexane NS ® | 1.8% |
| Monosodium stearoylglutamate | 0.2% |
| 3-Benzyloxypropyl(4,5-diphenylimidazol)-1-yl | 1.0% |
| Carbomer | 0.2% |
| Triethanolamine qs | pH = 7 |
| Preserving agents qs | |
| Fragrances qs | |
| Demineralized water qs | 100% |

This gel is applied to the skin, once or twice a day.

Anti-irritant Lotion for Preventing Hair Loss:

| | |
|---|---|
| 3-Benzyloxypropyl(4,5-diphenylimidazol)-1-yl | 1.0% |
| Propylene glycol | 30.0% |
| Ethyl alcohol | 40.5% |
| Water qs | 100% |

This lotion is applied once or twice a day, at a rate of 1 ml per application.

Thickened Lotion:

| | |
|---|---|
| 3-Benzyloxypropyl(4,5-diphenylimidazol)-1-yl | 1.0% |
| Kawaine | 2.0% |
| Hydroxypropylcellulose sold by the company | 3.5% |
| Hercules under the name Klucel G ® | |
| Ethyl alcohol qs | 100% |

This thickened lotion is applied once or twice a day.

Lotion:

| | |
|---|---|
| Chimexane NL ® | 0.50% |
| Cholesterol | 0.40% |
| Monosodium stearoylglutamate | 0.05% |
| 3-Benzyloxypropyl(4,5-diphenylimidazol)-1-yl | 0.50% |
| Preserving agents qs | |
| Colorants qs | |
| Fragrance qs | |
| Demineralized water qs | 100% |

This lotion is applied once or twice a day, at a rate of 1 ml per application.

Lotion:

| | |
|---|---|
| 3-Benzyloxypropyl(4,5-diphenylimidazol)-1-yl | 0.1% |
| Propylene glycol monomethyl ether | 20.0% |
| sold under the name Dowanol PM ® by | |
| the company Dow Chemical | |
| Hydroxypropylcellulose sold by the | 3.0% |
| company Hercules under the name | |
| Klucel G ® | |
| Ethyl alcohol | 40.0% |
| Water qs | 100% |

This thickened lotion is applied at a rate of 1 ml per application.

Day Cream:

| | |
|---|---|
| 3-Benzyloxypropyl(4,5-diphenylimidazol)-1-yl | 1.0% |
| Sucrose stearate | 4.0% |
| Stearyl alcohol | 2.0% |
| Cyclohexasiloxane | 9.0% |
| Mineral oil | 4.0% |
| Glycerol | 5.0% |
| Xanthan gum | 0.3% |
| Carbomer | 0.5% |
| Preserving agents | 0.3% |
| Fragrance | 0.3% |
| Water qs | 100% |

Care Fluid:

| | |
|---|---|
| 3-Benzyloxypropyl(4,5-diphenylimidazol)-1-yl | 1.0% |
| Stearyl alcohol | 0.4% |
| Sorbitan stearate | 1.5% |
| Glycerol | 5.0% |
| Xanthan gum | 0.2% |
| Carbomer | 0.1% |
| Cyclohexasiloxane | 7.0% |
| Preserving agents | 0.3% |
| Fragrance | 0.2% |
| Water qs | 100% |

Lotion:

| | |
|---|---|
| 3-Benzyloxypropyl(4,5-diphenylimidazol)-1-yl | 0.5% |
| Propylene glycol | 2.0% |
| Extract of cornflower | 0.1% |
| Preserving agents | 0.1% |
| PEG-60 hydrogenated castor oil | 0.4% |
| Fragrance | 0.1% |
| Water qs | 100% |

1-40 Cancelled.

41. A compound of the 3-alkyl(4,5-diphenylimidazol-1-yl) family corresponding to formula (I) below: 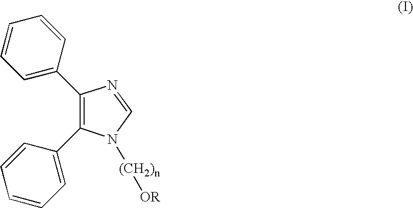
wherein
n is equal to 3, 4 or 5;
R represents, with the exception of an unsubstituted linear C₁-C₄ alkyl radical and/or an unsubstituted aryl radical, a linear or branched C₁-C₈ alkyl radical, optionally substituted with at least one aryl and/or aralkyl radical and/or a hydroxyl radical (—OH) and/or a radical —OR₁ and/or a radical —SR₁ and/or a radical —NR₁R₂ and/or a heterocycle, for which R₁ and R₂ represent a linear or branched C₁-C₄ alkyl radical,
an aryl or alkylaryl or arylalkyl radical or a heterocycle, optionally substituted with at least one alkyl radical and/or a hydroxyl radical (—OH) and/or a radical —OR₁, and/or a radical —SR₁ and/or a radical —NR₁R₂, for which R₁ and R₂ represent a linear or branched C₁-C₄ alkyl radical, a phenyl radical or a benzyl radical
or physiologically acceptable salts thereof or optical or geometrical isomers thereof.

42. A compound of formula (I) as defined in claim 41, wherein the compound is selected from the group consisting of
3-benzyloxypropyl(4,5-diphenylimidazol)-1-yl;
4-benzyloxybutyl(4,5-diphenylimidazol)-1-yl;
5-benzyloxypentyl(4,5-diphenylimidazol)-1-yl;
3-(2-tetrahydro-2H-pyranoxy)propyl(4,5 -diphenylimidazol)-1-yl;
4-(2-tetrahydro-2H-pyranoxy)butyl(4,5 -diphenylimidazol)-1-yl;
5-(2-tetrahydro-2H-pyranoxy)pentyl(4,5-diphenylimidazol)-1-yl;
3-(2-methoxyetboxy)propyl(4,5-diphenylimidazol)-1-yl;
4-(2-methoxyethoxy)butyl(4,5-diphenylimidazol)-1-yl;
5-(2-methoxyethoxy)pentyl(4,5-diphenylimidazol)-1-yl;
3-(2-ethoxyethoxy)propyl(4,5-diphenylimidazol)-1-yl;
4-(2-ethoxyethoxy)butyl(4,5-diphenylimidazol)-1-yl;
5-(2-ethoxyethoxy)pentyl(4,5-diphenylimidazol)-1-yl;
3-(2-propyloxyethoxy)propyl(4,5-diphenylimidazol)-1-yl;
4-(2-propyloxyethoxy)butyl(4,5-diphenylimidazol)-1-yl;
5-(2-propyloxyethoxy)pentyl(4,5-diphenylimidazol)-1-yl;
3-(methoxymethoxy)propyl(4,5-diphenylimidazol)-1-yl;
4-(methoxymethoxy)butyl(4,5-diphenylimidazol)-1-yl;
5-(methoxymethoxy)pentyl(4,5-diphenylimidazol)-1-yl;
3-(ethoxymethoxy)propyl(4,5-diphenylimidazol)-1-yl;
4-(ethoxymethoxy)butyl(4,5-diphenylimidazol)-1-yl;
5-(ethoxymethoxy)pentyl(4,5-diphenylimidazol)-1-yl;
3-(propyloxymethoxy)propyl(4,5-diphenylimidazol)-1-yl;
4-(propyloxymethoxy)butyl(4,5-diphenylimidazol)-1-yl;
5-(propyloxymethoxy)pentyl(4,5-diphenylimidazol)-1-yl;
3-(3-methoxypropyloxy)propyl(4,5-diphenylimidazol)-1-yl;
4-(3-methoxypropyloxy)butyl(4,5-diphenylimidazol)-1-yl;
5-(3-methoxypropyloxy)pentyl(4,5-diphenylimidazol)-1-yl;
3-(3-ethoxypropyloxy)propyl(4,5-diphenylimidazol)-1-yl;
4-(3-ethoxypropyloxy)butyl(4,5-diphenylimidazol)-1-yl;
5-(3-ethoxypropyloxy)pentyl(4,5-diphenylimidazol)-1-yl;
3-(3-propyloxypropyloxy)propyl(4,5-diphenylimidazol)-1-yl;
4-(3-propyloxypropyloxy)butyl(4,5-diphenylimidazol)-1-yl;
5-(3-propyloxypropyloxy)pentyl(4,5-diphenylimidazol)-1-yl;
3-(2,3,4-trimethoxybenzyloxy)propyl(4,5-diphenylimidazol)-1-yl;
4-(2,3,4-trimethoxybenzyloxy)butyl(4,5-diphenylimidazol)-1-yl;
5-(2,3,4-trimethoxybenzyloxy)pentyl(4,5-diphenylimidazol)-1-yl;
3-(3,4-dimethoxybenzyloxy)propyl(4,5-diphenylimidazol)-1-yl;
4-(3,4-dimethoxybenzyloxy)butyl(4,5-diphenylimidazol)-1-yl;
5-(3,4-dimethoxybenzyloxy)pentyl(4,5-diphenylimidazol)-1-yl;
3-(3-methoxybenzyloxy)propyl(4,5-diphenylimidazol)-1-yl;
4-(3-methoxybenzyloxy)butyl(4,5-diphenylimidazol)-1-yl;
5-(3-methoxybenzyloxy)pentyl(4,5-diphenylimidazol)-1-yl;
3-(4-methoxybenzyloxy)propyl(4,5-diphenylimidazol)-1-yl;
4-(4-methoxybenzyloxy)butyl(4,5-diphenylimidazol)-1-yl;
5-(4-methoxybenzyloxy)pentyl(4,5-diphenylimidazol)-1-yl;
3-(3-pyridylmethoxy)propyl(4,5-diphenylimidazol)-1-yl;
4-(3-pyridylmethoxy)butyl(4,5-diphenylimidazol)-1-yl;
5-(3-pyridylmethoxy)pentyl(4,5-diphenylimidazol)-1-yl;
3-(4-benzyloxy-3-methoxybenzyloxy)propyl(4,5-diphenylimidazol)-1-yl;
4-(4-benzyloxy-3-methoxybenzyloxy)butyl(4,5-diphenylimidazol)-1-yl;
5-(4-benzyloxy-3-methoxybenzyloxy)pentyl(4,5-diphenylimidazol)-1-yl;
3-(1-naphthalenemethoxy)propyl(4,5-diphenylimidazol)-1-yl;
4-(1-naphthalenemethoxy)butyl(4,5-diphenylimidazol)-1-yl;
5-(1-naphthalenemethoxy)pentyl(4,5-diphenylimidazol)-1-yl; physiologically acceptable salts thereof; optical isomers; and geometrical isomers thereof.

43. A compound of formula (I) as defined in claim 41, wherein n is equal to 3 or 4.

44. A compound of formula (I) as defined in claim 41, wherein R denotes a benzyl radical.

45. A compound of formula (I) as defined in claim 41, wherein said compound is selected from the group consisting of
3-benzyloxypropyl(4,5-diphenylimidazol)-1-yl and
4-benzyloxybutyl(4,5-diphenylimidazol)-1-yl.

46. A process for preparing the compounds of formula (I) as defined in 41, wherein said process consists essentially of:
a) dissolving 4,5-diphenylimidazole in an aprotic organic solvent; wherein said aprotic organic solvent is selected from the group consisting of acetonitrile, acetone, tetrahydrofuran, dimethylformamide, and mixtures thereof;
b) adding an organic or mineral base in an amount of between 1 and 10 equivalents;
c) adding an alkyl halide in an amount of between 1 and 5 equivalents;
d) heating to a temperature of between 50° C. and 85° C. for a period of between 6 hours and 48 hours;
e) evaporating the reaction medium to dryness under vacuum after cooling to room temperature;
f) dissolving the residue in water;
g) extracting the aqueous solution with an organic solvent selected from the group consisting of ethyl acetate, dichloromethane, diethyl ether, and mixtures thereof;
h) drying and evaporating the organic phase to dryness;
i) optionally purifying the residue.

47. A preparation process according to claim 46, wherein the aprotic organic solvent of said dissolving step, a), is acetonitrile.

48. A preparation process according to claim 46, wherein said adding, b), comprises adding a mineral base.

49. A preparation process according to claim 46, wherein said adding, b), comprises adding a mineral base, and wherein said mineral base is selected from the group consisting of potassium carbonate, calcium carbonate, sodium carbonate, and mixtures thereof.

50. A preparation process according to claim 46, wherein said adding, b), comprises adding a mineral base, and wherein said mineral base is potassium carbonate.

51. A preparation process according to claim 46, wherein said adding of the organic or mineral base occurs in an amount of between 1 and 3 equivalents.

52. A preparation process according to claim 46, wherein said adding an alkyl halide occurs in an amount of 1.5 equivalents.

53. A preparation process according to claim 46, wherein said heating, d), occurs at a temperature of between 70° C. and 85° C.

54. A preparation process according to claim 46, wherein said heating, d), occurs for a period of between 12 hours and 30 hours.

55. A preparation process according to claim 46, wherein said heating, d), occurs for a period of 24 hours.

56. A preparation process according to claim 46, wherein said extracting, g), the organic solvent is ethyl acetate.

57. A process for preparing the compounds of formula (I) as defined in 41, which consists essentially of:
a) dissolving 4,5-diphenylimidazole in a dipolar aprotic solvent; wherein the aprotic solvent is selected from the group consisting of dimethylformamide, dimethyl sulphoxide, dimethylacetamide, and mixtures thereof;
b) adding under an inert atmosphere an organic or mineral base in an amount of between 0.9 and 1.5 equivalents;
c) stirring the reaction medium at a temperature of between 25° C. and 100° C. for a period of between 30 minutes and 10 hours;
d) adding an alkyl halide in an amount of between 1 and 3 equivalents;
e) adding potassium iodide or sodium iodide in an amount of between 1 and 3 equivalents;
f) heating at a temperature of between 25° C. and 100° C. for a period of between 5 hours and 24 hours;
g) taking up the reaction medium in an organic solvent selected from the group consisting of dichloromethane, chloroform, and mixtures thereof;
h) washing the organic phase with water and bicarbonate, drying it and then evaporating it to dryness;
k) optionally purifying the residue.

58. A preparation process according to claim 57, wherein in said dissolving, a), the aprotic organic solvent is dimethylformamide.

59. A preparation process according to claim 57, wherein said adding, b) comprises adding a mineral base.

60. A preparation process according to claim 59, wherein the mineral base is selected from the group consisting of sodium hydride, butyllithium, and mixtures thereof.

61. A preparation process according to claim 59, wherein the mineral base and is sodium hydride.

62. A preparation process according to claim 57, wherein said adding, b), under an inert atmosphere an organic or mineral base occurs in an amount between 1.0 and 1.1 equivalents.

63. A preparation process according to claim 57, wherein said stirring, c), occurs at a temperature of 50° C., for a period of between 1 hour and 2 hours.

64. A preparation process according to claim 57, wherein said adding an alkyl halide occurs in an amount between 1 and 2 equivalents of alkyl halide.

65. A preparation process according to claim 57, wherein said adding, e), occurs in an amount between 1 and 2 equivalents of potassium iodide or sodium iodide.

66. A preparation process according to claim 57, wherein said heating, f), occurs at a temperature of between 40° C. and 60° C., for a period of between 10 hours and 20 hours.

67. A preparation process according to claim 57, wherein the organic solvent employed in said taking up the reaction medium, g), is dichloromethane.

68. A composition comprising at least one compound of formula (I) as defined according to claim 41.

69. A cosmetic or pharmaceutical composition, which comprises the composition as claimed in claim 68.

70. A cosmetic composition according to claim 69, wherein the cosmetic composition comprises at least one compound of formula (I) in an amount representing from 0.001% to 20% of the total weight of the composition.

71. A composition according to claim 68, which further comprises an agent which is selected from the group consisting of an antibacterial agent, an antiparasitic agent, an antifungal agent, an antiviral agent, an antiinflammatory agent, an antipruriginous agent, an anaesthetic, a keratolytic agent, a free-radical scavenger, an antiseborrhoeic agent, an antidandruff agent, an antiacne agent, an agent for reducing cutaneous differentiation, an an agent for reducing cutaneous proliferation, an agent for reducing cutaneous pigmentation, an agent for improving the activity on regrowth of the hair, a hair loss reduction agent, an plant extract, a bacterial extract, and mixtures thereof.

72. A composition according to claim 71, wherein the antiinflammatory agent is a steroidal antiinflammatory agent or a non-steroidal antiinflammatory agent.

73. A cosmetic or pharmaceutical composition, which comprises, in a cosmetically or pharmaceutically acceptable medium,
at least one compound of the 3-alkyl(4,5-diphenylimidazol-1-yl) family corresponding to formula (I) as defined in claim 41 and
at least one product with an irritant side effect.

74. A composition according to claim 73, wherein the product with an irritant side effect is selected from the group consisting of an ionic surfactant; a nonionic surfactant; a preserving agent; an organic solvent; an active agent which is selected from the group consisting of an α-hydroxy acid, a β-hydroxy acid, an α-keto acid, a β-keto acid, a retinoid, an anthralin, an anthranoid, a peroxide, a minoxidil, a lithium salt, an antimetabolite, vitamin D, and derivatives thereof; a hair dye; a hair colorant; a fragrant alcoholic solution; an antiperspirant; a hair-removing active agent; a permanent-waving active agent; a depigmenting active agent; and mixtures thereof.

75. A skin disturbance treating process, which comprises:
applying to the skin and/or scalp and/or mucosa of a mammal a preparation of a calmative composition comprising at least one compound of the 3-alkyl(4,5-diphenylimidazol-1-yl) family corresponding to formula (I) as defined in claim 41 and a physiologically acceptable medium.

76. A process according to claim 75, wherein said skin disturbance is selected from the group consisting of sensitive skin, discomfort, tautness, itching, irritation, redness, a heat sensation, an inflammatory sensation, and combinations thereof.

77. A process according to claim 75, wherein said applying occurs when the mammal experiences one or more symptoms selected from the group consisting of sensitive skin, irritable skin, reactive skin, intolerant skin and/or one or more symptoms selected from the group consisting of sensitive scalp, irritable scalp, reactive scalp, intolerant scalp and/or one or more symptoms selected from the group consisting of sensitive mucous membranes, irritable mucous membranes, reactive mucous membranes, intolerant mucous membranes, and combinations thereof.

78. A process according to claim 75, wherein said applying occurs when the skin and/or scalp and/or mucosal membrane of the mammal experiences one or more symptoms selected from the group consisting of stinging, tingling, itching, pruritis, inflammation, discomfort, and tautness, and combinations thereof.

79. A hair loss reduction method, which comprises:
applying to the scalp and/or skin of a human male a cosmetic or pharmaceutical composition comprising at least one compound of the 3-alkyl(4,5-diphenylimidazol-1-yl) family of formula (I) as defined in claim 41 and a physiologically acceptable medium;
leaving the cosmetic or pharmaceutical composition in contact with the scalp and/or skin of the human male; and optionally
rinsing the skin of a human.
